(19) United States
(12) Patent Application Publication
(10) Pub. No.: US 2024/0074788 A1
(43) Pub. Date: Mar. 7, 2024

(54) DEVICES AND SYSTEMS FOR USE IN LAPAROSCOPIC SURGERY

(71) Applicant: Axcess Instruments IP Holding Company, Tyler, TX (US)

(72) Inventors: Michael J. Norton, Tyler, TX (US); Noel D. Ischy, Tyler, TX (US); Matthew Smith, Tyler, TX (US)

(73) Assignee: Axcess Instruments IP Holding Company, Tyler, TX (US)

(21) Appl. No.: 18/505,028

(22) Filed: Nov. 8, 2023

Related U.S. Application Data
(63) Continuation-in-part of application No. 18/213,741, filed on Jun. 23, 2023.
Application No. 18/213,741 is a continuation-in-part of application No. 17/702,193, filed on Mar. 23, 2022, now abandoned.
(60) Provisional application No. 63/424,064, filed on Nov. 9, 2022.
(60) Provisional application No. 63/165,045, filed on Mar. 23, 2021.
(60) Provisional application No. 63/424,064, filed on Nov. 9, 2022.

(51) Int. Cl. A61B 17/34; A61B 1/00; A61B 1/015; A61B 1/018; A61B 1/05; A61B 1/06; A61B 1/12; A61B 1/313

(52) U.S. Cl. CPC A61B 17/3423; A61B 1/00091; A61B 1/015; A61B 1/018; A61B 1/05; A61B 1/0676; A61B 1/0684; A61B 1/126; A61B 1/3132; A61B 17/3474

(57) ABSTRACT

In accordance with at least one aspect of this disclosure, a wash nozzle structure for diverting a wash fluid to a surgical instrument during a laparoscopic procedure, can be configured to direct the wash fluid to spray along a spray axis, where the spray axis is angled relative to a plane of a lens of an imaging device of the surgical instrument.

### CROSS REFERENCE TO RELATED APPLICATIONS

This application claims priority to U.S. Provisional Patent Application No. 63/424,064, Filed Nov. 9, 2022, and is a continuation in part of U.S. patent application Ser. No. 18/213,741, filed Jun. 23, 2023, which is a continuation-in-part of U.S. patent application Ser. No. 17/702,193, filed Mar. 23, 2022, which claims priority to and the benefit of U.S. Provisional Patent Application No. 63/165,045, filed Mar. 23, 2021. The entire content of these applications is incorporated by reference herein.

### TECHNICAL FIELD

The subject invention is directed to surgery, and more particularly, to an anchored access port having an attachment to support accessories, such as imaging and lighting devices, used in minimally invasive surgical procedures performed within the abdominal cavity of a patient, including, but not limited to, laparoscopic surgical procedures.

### BACKGROUND

Laparoscopic or “minimally invasive” surgery is widely practiced in multiple specialties of medicine, and is commonplace in the performance of procedures such as cholecystectomies, appendectomies, hernia repair and nephrectomies, for example. Benefits of such procedures include reduced trauma to the patient, reduced opportunity for infection, and decreased recovery time.

Such procedures within the abdominal (peritoneal) cavity are typically performed through a device known as a trocar or cannula, which facilitates the introduction of laparoscopic instruments into the abdominal cavity of a patient while under vision through a laparoscope, which require an organized operational space to function together or independently. Laparoscopes for performing these procedures have been used in various forms for nearly 100 years. They are significant in size and require an accompaniment of support equipment for effective imaging of the operative site. It would be useful therefore, to reduce the size and format of this physical imaging instrument while increasing its visual, lighting and format capabilities. This would further enable ancillary surgical instruments.

Such conventional methods and systems have generally been considered satisfactory for their intended purpose. However, there is still a need in the art for improved medical devices. The present disclosure provides a solution for this need.

### SUMMARY

In accordance with at least one aspect of this disclosure, a surgical access device assembly for performing laparoscopic surgery can include first surgical instrument and an access port defining a body, the body having a central bore defined therethrough configured to receive the first surgical instrument. The first surgical instrument includes a longitudinal portion extending along a longitudinal axis of the first surgical instrument and a radial extension extending from a distal end of the longitudinal portion and an imaging device disposed in the radial extension configured to image a surgical site.

A wash channel can be disposed in and/or defined through the longitudinal portion and in fluid communication between a wash channel inlet and a wash channel outlet to allow a wash flow to flow from the wash channel inlet to the wash channel outlet. A wash nozzle structure can be included radially inward of the imaging device and defining a flow shaping cavity and a wash nozzle outlet in fluid communication with the wash channel outlet, the wash nozzle structure configured to direct the wash flow from the wash channel outlet, through the flow shaping cavity, to the nozzle outlet to shape the wash flow to effuse from the nozzle outlet such that the wash flow is shaped to sweep across the imaging device to wash the imaging device during the laparoscopic procedure.

In embodiments, the wash nozzle structure can be configured to increase a flow velocity of the wash flow at the wash nozzle outlet and shape the wash flow to effuse from the wash nozzle structure at least partially along a spray axis, wherein the spray axis is angled relative to an axis of the radial extension.

In embodiments, the wash nozzle structure can include a nozzle structure body defining a central axis, a proximal face, a distal face, and a bowl recessed into proximal face of the body. The bowl can include a first angled face defined in a first plane, a second angled face defined in a second plane, a third angled face defined in a third plane, and a fourth face defined in a fourth plane co-planar with the proximal face and the distal face. In embodiments, the first plane, second plane, third plane, and fourth plane can be different from one another. The first angled face, the second angled face, and the third angled face can be arranged to funnel the wash fluid to the fourth face to direct the wash fluid to spray at least partially along a spray axis. In embodiments, the spray axis can be parallel to the central axis of the body or angled relative thereto. In certain embodiments, each of the first angled face, the second angled face and the third angled face can have a concave curvature.

In embodiments, the first surgical instrument can also include an insufflation channel disposed in and/or defined through the longitudinal portion and in fluid communication between an insufflation channel inlet and an insufflation channel outlet to allow insufflation gas to flow from the insufflation channel inlet to the insufflation channel outlet and into a patient's abdominal cavity.

In embodiments, the first surgical instrument can further include a lighting device disposed at a distal end of the radial extension, radially outward of the imaging device, configured to illuminate the surgical site within a surgical cavity. The lighting device can include at least one illuminator (e.g., an LED).

In certain embodiments, the access port can further include an access slot defined in the body configured to receive a second surgical instrument. In embodiments, the second surgical instrument can be one of a laser device, a measuring device, a laser measuring device, a signal transmitting device, a signal receiving device, a signal processing device, a memory storage device, a wiring device, a servo driven device, a gear device, a separate irrigation device, and/or a suction device.

In accordance with at least one aspect of this disclosure, access port for accommodating at least one surgical instrument during laparoscopic or minimally invasive surgery, can include a body having a proximal end portion and distal end portion, the body defining a central axis and extending from the proximal portion to the distal end portion along the central axis. A central bore can be defined through the body and extending from a proximal face defined in the proximal end portion to a first distal face defined in the distal end portion, the central bore configured to accept a first surgical instrument. In embodiments, the access portion can also include an access slot defined though the body and extending from the proximal face to a second distal face defined in the distal end portion.

In certain embodiments, the access slot can include an arcuate (e.g., crescent) entrance defined in the proximal face radially outboard of the central bore and a circular exit defined in the second distal face. The circular exit can be outboard of the central bore. The access slot can be configured to accept a second surgical instrument and allow for sliding movement of the surgical instrument within the access slot along a curvature of the arcuate entrance of the slot.

The body can define a proximal frustoconical portion and a distal elbow portion extending from the proximal frustoconical portion. The first distal face can be defined in the distal elbow portion out of plane with the proximal face, and the second distal face can be defined in the distal elbow portion co-planar with the proximal face. The central bore can be defined partially in the first distal face and partially in the second distal face.

In embodiments, the distal elbow portion can define a concave anchoring region configured to anchor the body with respect to an interior surface of a patient's abdominal wall, wherein at least a portion of the proximal frustoconical portion is configured to be held outside of the patients abdominal wall and at least a portion of the distal elbow portion is configured to be held within the patient's abdominal wall with the body inserted into a surgical access site for allowing access to the surgical cavity through the access port body. In embodiments, the anchoring region can include an anchoring shape at a distal curved longitudinal surface oriented away from the body, and the anchoring shape can be disposed relatively opposite to a radial orientation of the boot tip.

In certain embodiments, the body can be formed of a flexible material configured to allow for manipulation of the body with insertion of the first and/or second surgical instrument through the central bore and/or the access slot. In certain embodiments, the flexible material can be or can include silicone. In embodiments, a valve flap can be defined in the access slot closer to the distal end portion of the body than the proximal end portion.

In embodiments, the body can be at least partially boot shaped, where the first distal face extends radially outward from the proximal frustoconical to form a toe portion and the second distal face extends axially from the proximal frustoconical portion to form a heel portion.

In accordance with at least one aspect of this disclosure, a wash nozzle structure for diverting a wash fluid to a surgical instrument during a laparoscopic procedure, can be configured to direct the wash fluid to spray along a spray axis, where the spray axis is angled relative to a plane of a lens of an imaging device of the surgical instrument. Embodiments of the wash nozzle structure can be as described herein.

In certain embodiments, each of the first angled face, the second angled face and the third angled face have a concave curvature. In certain embodiments, the first angled face and the third angled face can be angled relative to the fourth face at a 90 degree angle. In certain embodiments, the first angled face and the third angled face are angled relative to the fourth face at an angle greater than 90 degrees.

In certain embodiments, the second angled face can be angled relative to the fourth face at an angle greater than 90 degrees.

In certain embodiments, the first angled face and the third angled face can be angled relative to the second angled face at a 90 degree angle.

In certain embodiments, the first angled face and the third angled face can be angled relative to the second angled face at an angle greater than 90 degrees.

In accordance with at least one aspect of this disclosure, a kit can include an access port as described herein and a surgical instrument as described herein for inserted into the access port. The access port and the surgical instrument can be included in a sterile package.

These and other features of the systems and methods of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, other embodiments thereof will be described in detail herein below with reference to certain figures, wherein:

FIG. 1 is a perspective view of an assembly in accordance with this disclosure, showing a surgical instrument inserted into an access port;

FIG. 2 is a side elevation view of the assembly of FIG. 1, showing the assembly inserted into a surgical site (e.g., an abdominal wall of a patient) and a breakaway cross section of the assembly taken along section line 2-2 in FIG. 1;

FIG. 3 is a front perspective view of the access port of FIG. 1;

FIG. 4 is a rear perspective view of the access port of FIG. 1;

FIG. 5A is a cross-sectional view of the access port of FIG. 1, taken along section 5A-5A shown in FIG. 3;

FIG. 5B is an enlarged partial view of the section shown in FIG. 5, showing a valve flap;

FIG. 6 is a bottom up view of the assembly shown in FIG. 1, showing an underside of the surgical instrument and an underside of the access port;

FIG. 7 is a perspective view of an embodiment of a wash nozzle structure;

FIG. 8 is a plan view of the wash nozzle structure of FIG. 7;

FIG. 9 is a front elevational view of the wash nozzle structure of FIG. 8;

FIG. 10 is a perspective view of another embodiment of a wash nozzle structure;

FIG. 11 is a plan view of the wash nozzle structure of FIG. 10;

FIG. 12 is a front elevational view of the wash nozzle structure of FIG. 10;

FIG. 13 is a bottom perspective view of the assembly of FIG. 1, the assembly inserted into a surgical site (e.g., an abdominal wall of a patient), showing irrigation of the surgical instrument via the wash nozzle structure.

### DETAILED DESCRIPTION

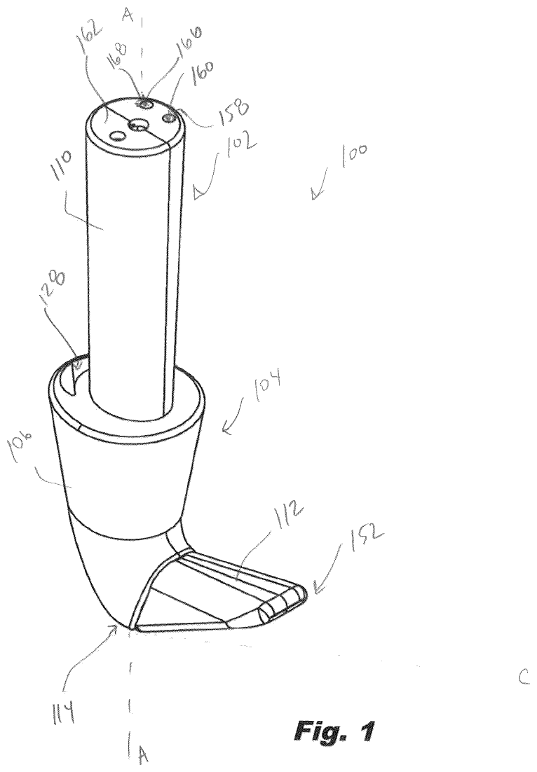

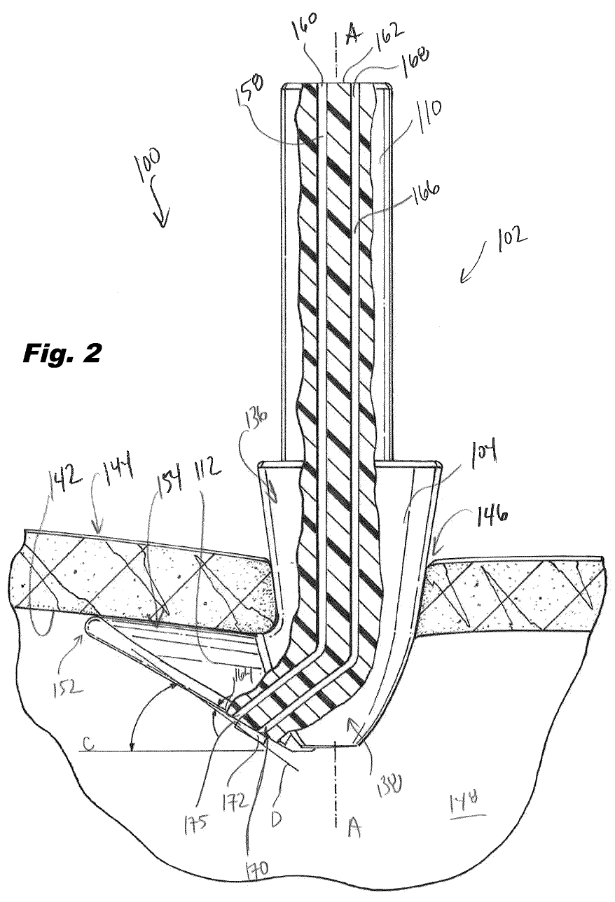

Reference will now be made to the drawings wherein like reference numerals identify similar structural features or aspects of the subject disclosure. For purposes of explanation and illustration, and not limitation, an illustrative view of an embodiment of an assembly in accordance with the disclosure is shown in FIG. 1 and is designated generally by reference character 100. Other embodiments and/or aspects of this disclosure are shown in FIGS. 2-13. Certain embodiments described herein can be used to provide access to a surgical cavity for providing imaging of and lighting to the surgical site, and further provide means for washing the imaging device during a laparoscopic procedure.

As shown in FIGS. 1-5B, in accordance with at least one aspect of this disclosure, a surgical access device assembly 100 for performing laparoscopic surgery includes a surgical instrument 102 and an access port 104 defining a body 106 and a central bore 108 therethrough configured to receive the surgical instrument 102. The surgical instrument 102 can include, as shown, a longitudinal portion 110 extending along a longitudinal axis A of the surgical instrument 102 and a radial extension 112 extending from a distal end 114 of the longitudinal portion 110, which can include at least an imaging device 116 and a lighting device 118, as described further herein below.

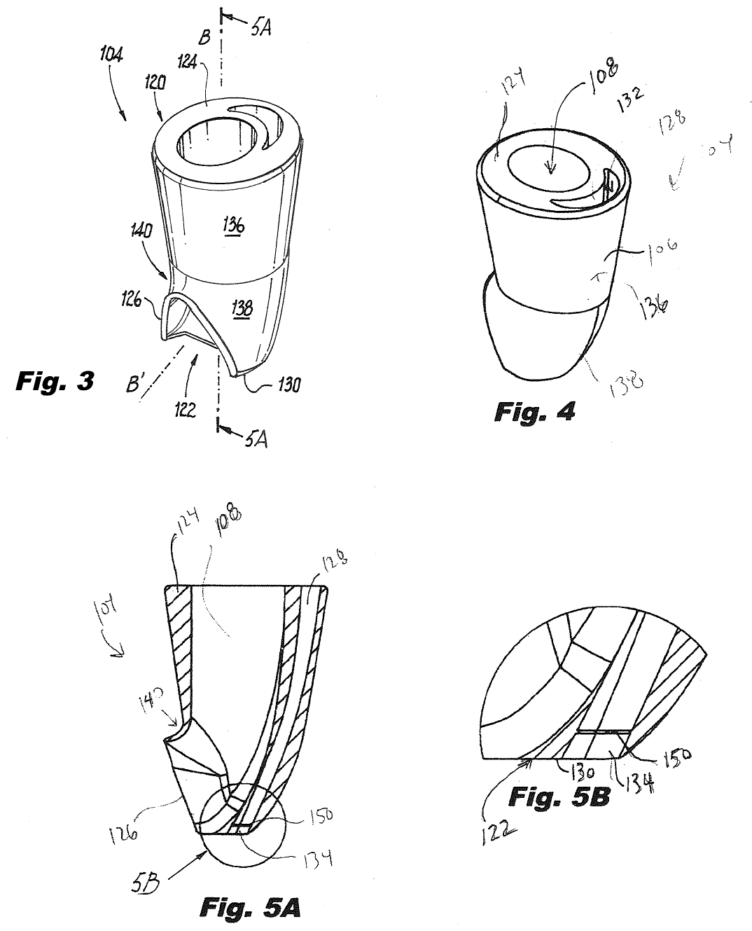

As shown in FIGS. 3-5B, the body 106 of the access port can include a proximal end portion 120 and distal end portion 122, and can define a central body axis B extending from the proximal end portion 102 to the distal end portion 22. The central bore 108 can be defined through the body 106 extending from a proximal face 124 defined in the proximal end portion 120 to a first distal face 126 defined in the distal end portion 122. In embodiments, the access port 104 can also include an access slot 128 defined though the body 106 and extending from the proximal face 124 to a second distal face 130 defined in the distal end portion 122 of the access port body 106, configured to receive a second surgical instrument (the second surgical instrument not shown).

In embodiments, the second surgical instrument can be one of a laser device, a measuring device, a laser measuring device, a signal transmitting device, a signal receiving device, a signal processing device, a memory storage device, a wiring device, a servo driven device, a gear device, a separate irrigation device, and/or a suction device.

In certain embodiments, the access slot 128 can include an arcuate (e.g., crescent) entrance 132 defined in the proximal face 124 radially outboard of the central bore 108 and a circular exit 134 defined in the second distal face 130. The circular exit 134 can be outboard of the central bore 108. The access slot 128 can be configured to accept the second surgical instrument and allow for sliding movement of the surgical instrument within the access slot, e.g., along a curvature of the arcuate entrance 132 of the slot 128. The access slow 128 thus allows for the second surgical instrument to be inserted through the access port 104 and manipulated within the surgical site while the surgical instrument 102 is inserted through the central bore 108.

The body 106 of the access port 104 can define a proximal frustoconical portion 136 and a distal elbow portion 138 extending from the proximal frustoconical portion 136. The first distal face 126 can be defined in the distal elbow portion 138 out of plane with the proximal face 124, and the second distal face 130 can be defined in the distal elbow portion 138 co-planar with the proximal face 124.

In embodiments, the distal elbow portion 138 can define a concave anchoring region 140 configured to anchor the body 106 with respect to an interior surface 142 of a patient's abdominal wall 144, for example as shown in FIG. 2. At least a portion of the proximal frustoconical portion 136 is configured to be held outside of the patient's abdominal wall 144 and at least a portion of the distal elbow portion 138 is configured to be held within the patient's abdominal wall 144 when the access port body 106 is inserted into a surgical access site 146 for allowing access to a surgical cavity 148 through the access port body 106.

In certain embodiments, the body 106 can be formed of a flexible material configured to allow for manipulation of the body 106 with insertion of the first and/or second surgical instrument 102 through the central bore 108 and/or the access slot 128. In certain embodiments, the flexible material can be or can include silicone. In embodiments, a valve flap 150 can be defined in the access slot 128 closer to the second distal face 130 of the distal end portion 122 of the body 106 than the proximal end portion 120, for example, as shown in FIG. 5B.

In embodiments, e.g., as shown, the body 106 can be at least partially boot shaped, where the first distal face 126 extends outward from the proximal frustoconical 136 at an angle to form a toe portion along axis B′ and the second distal face 130 extends axially from the proximal frustoconical portion 136 to form a heel portion along axis B. With the first surgical instrument 102 inserted into the central bore 108, a distal end 152 of the radial extension 112 can become the toe portion of the boot shape, and the distal end portion 122 of the access port body can become the heel portion of the boot shape. The concave anchoring region 140 of the access port body 106 anchors the access port 104 in the abdominal wall 144 and holds an outer face 154 of the radial extension 112 of the first surgical instrument 102 against the inner surface 142 of the abdominal wall 144, as shown in FIG. 2. In doing so, an inner face 156 of the radial extension 112 is angled relative to the horizontal C, e.g., as shown in FIGS. 1 and 2.

Referring now to FIGS. 1, 2 and 6, in embodiments, the first surgical instrument 102 can include the imaging device 116 disposed in the radial extension 112 configured to image the surgical site, and the lighting device 118 can be disposed at or near the distal end 152 of the radial extension 112, radially outward of the imaging device configured to illuminate the surgical site within a surgical cavity 148. In embodiments, the lighting device 118 can include at least one illuminator 156 (e.g., an LED), or a plurality of illuminators 156.

The first surgical instrument 102 can further include disposed therein or defined therethrough, an insufflation channel 158 in fluid communication between and extending from an insufflation channel inlet 160 defined in a proximal face 162 of the longitudinal portion 110 to an insufflation channel outlet 164 defined in the radial extension 112 to deliver insufflation gas to the patient's abdominal cavity 148.

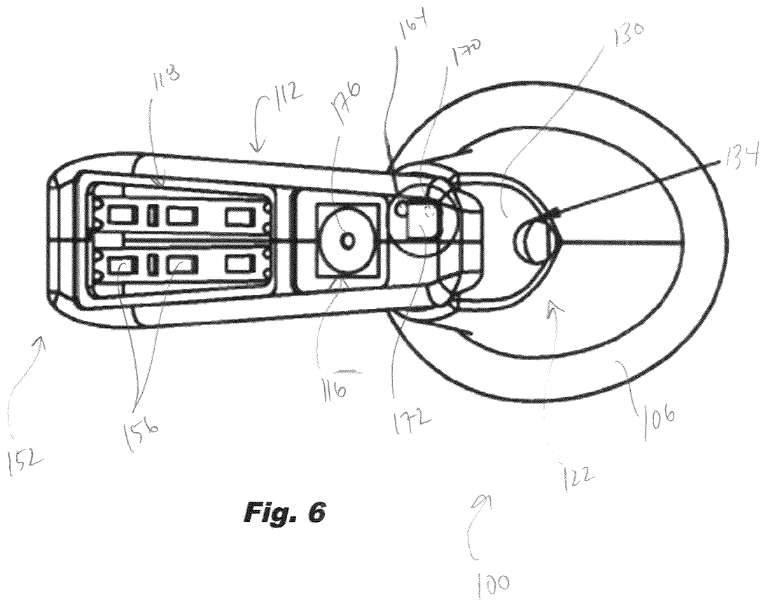

The first surgical instrument 102 can further include a wash channel 166 defined through and/or disposed in the first surgical instrument 102, in fluid communication between and extending from a wash channel inlet 168 defined in the proximal face 162 of the longitudinal portion to a wash channel outlet 170 defined in the radial extension 112. In FIG. 6, the outlet 170 is shown in phantom as it is obscured by a wash nozzle structure 172.

The wash nozzle structure 172 is operatively connected to the radial extension 112, proximate the imaging device 116 (e.g., radially inward of) and configured to disperse a wash fluid 174 over the imaging device 116 to wash the imaging device 116 during the laparoscopic procedure. For example, the wash nozzle structure 172 can define a flow shaping cavity 173, where the wash channel outlet 170 exits into the the flow shaping cavity 173. The wash nozzle structure 172 defines a wash nozzle outlet 175 having a height H that is less than a width W of the nozzle structure 172, such that the wash nozzle outlet 175 is configured to act as a reducing portion to increase a flow velocity of the wash flow at the wash nozzle outlet 175. In certain embodiments, the height H of the nozzle outlet 175 can be about ¼ W, or less. In embodiments, the wash nozzle structure 172 can be configured, in conjunction with the reducing portion of the nozzle outlet 175, such that the wash nozzle structure 170 directs the wash fluid 174 to spray along a spray axis D, where the spray axis D of the wash nozzle structure 172 is angled relative to the plane of the lens 176 so as to impinge on the lens 176. In certain embodiments, the spray axis D can be parallel to a plane of the imaging device where the wash fluid 174 is configured to fan out from the nozzle outlet 175 and sweep along imaging device 116.

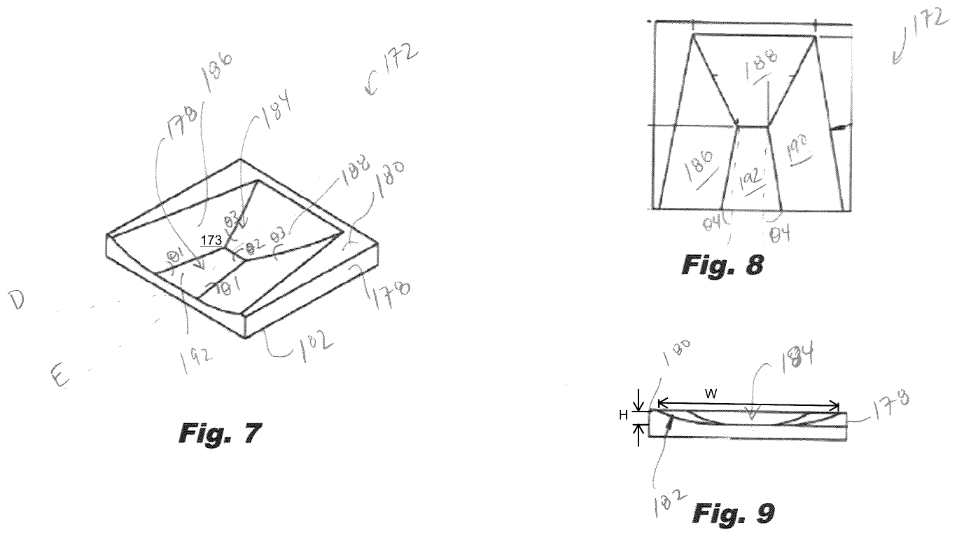

Now referring to FIGS. 7-12, the wash nozzle structure 172 will be described in further detail. The wash nozzle structure 172 can be configured and adapted (e.g., shaped and positioned relative to the exit of the wash channel outlet 170) to alter a force vector of the wash fluid 174 exiting the wash channel 166 at the outlet 170. The wash nozzle structure 172 can be configured to force the wash fluid centrally into a lower surface 178 of the nozzle structure (e.g., the bowl or flow shaping cavity 173 as described below), which then forces the wash fluid 174 outward (e.g., both along the spray axis and outward in a fan shape) over the imaging device 116 and, in embodiments, slightly upwards into the imaging device 116 to wash the lens 176 of the imaging device 116, e.g., as illustrated with spray axis D in FIG. 7 relative to a central axis E of the nozzle structure 172.

As shown in FIGS. 7-9, the wash nozzle structure 172 can include a nozzle structure body 178 defining a central axis E, a proximal face 180, a distal face 182, and a bowl 184 recessed into proximal face of the body 178. The body 178 can be generally rectangular, or in certain embodiments, largely square. The bowl 184 can include a first angled face 186 defined in a first plane, a second angled face 188 defined in a second plane, a third angled face 190 defined in a third plane, and a fourth face 192 defined in a fourth plane co-planar with the proximal face 180 and the distal face 180. In embodiments, the first plane, second plane, third plane, and fourth plane can be different from one another, e.g., out of plane with respect to one another. The first angled face 186, the second angled face 188, and the third angled face 190 can be arranged to funnel the wash fluid 174 to the fourth face 192 (e.g., a bottom of the flow shaping cavity 173) to direct the wash fluid 174 to spray at least partially along the spray axis D. In certain embodiments, each of the first angled face 186, the second angled face 188, and the third angled face 190 can have a concave curvature, e.g., to form the bowl shape as shown in FIG. 9.

Still with reference to FIGS. 7-9, the first angled face 186 and the third angled face 190 can be angled relative to the fourth face 192 at an angle θ1 greater than 90 degrees. The second angled face 188 can be angled relative to the fourth face 192 at an angle θ2 greater than 90 degrees. The first angled face 186 and the third angled face 190 can be angled relative to the second angled face 188 at an angle θ3 greater than 90 degrees. The first and third angled faces can diverge from the central axis by an angle θ4, which can be about 10 degrees.

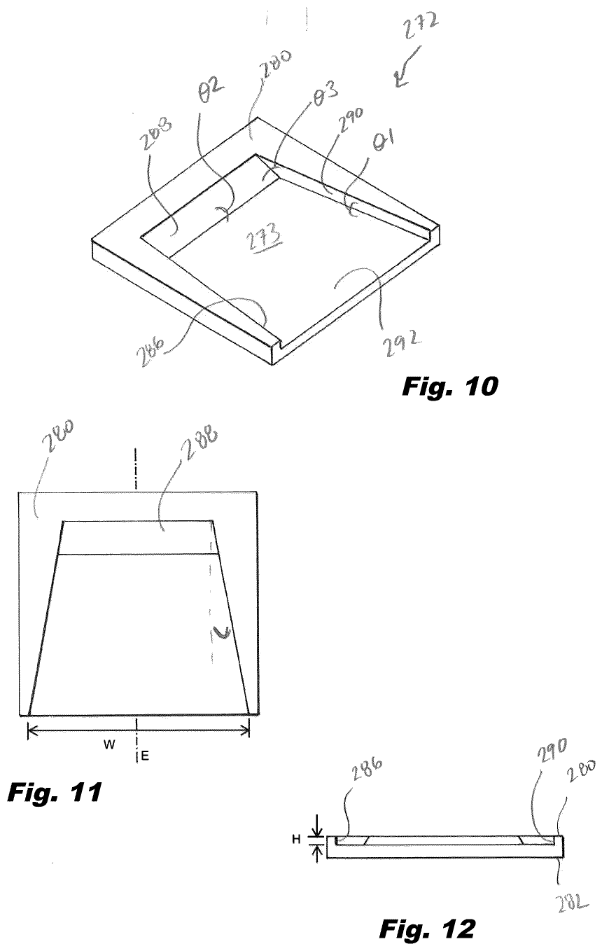

With reference to FIGS. 10-12, the first angled face 286 and the third angled face 290 can be angled relative to the fourth face 292 at an angle θ1 of about or equal to 90 degrees. The second angled face 288 can be angled relative to the fourth face 292 at an angle θ2 greater than 90 degrees. In certain embodiments, the first angled face 286 and the third angled face 290 can be angled relative to the second angled face 288 at an angle θ3 of about or equal to 90 degrees. In embodiments, the first and third angled faces 286, 290 can diverge from the central axis E by an angle of about 10 degrees, e.g., as shown in FIG. 11.

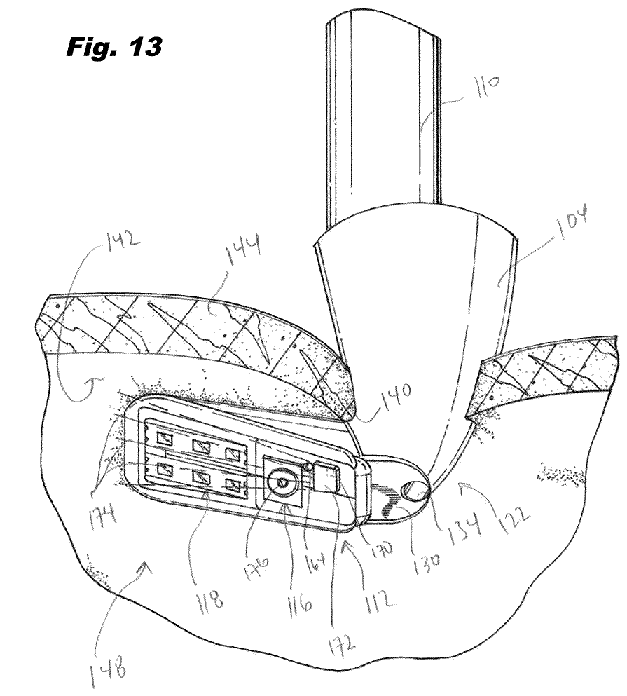

FIG. 13 shows a perspective view of the assembly 100 inserted into the surgical site, with a vantage point below the horizontal and looking upwards at the radial extension 112 during the laparoscopic procedure. Wash fluid 174 is shown being ejected from the nozzle structure 172 and onto the lens 176 of the imaging device 116. It is also possible that the wash fluid is spread across the one or more illuminators 156 of the lighting device 118. Directing the wash fluid 174 across the many components of the radial extension 112, especially the imaging device 116, using embodiments of the wash nozzle structure 172, 272 allows for in situ cleaning of the imaging device 116 during the surgical procedure, and without removal of the surgical instrument 102 from the surgical site. Embodiments of the wash nozzle structure 172, 272 (e.g., a flow diverter) can be configured to direct the flow of wash fluid 174 to directly impinge onto the imaging device 116 with enough force to wash and clean any debris from the imaging device lens 176 for improved visibility during the surgical procedure.

In certain embodiments, the wash nozzle structure can be attached or operatively connected to the first surgical instrument, e.g., using adhesive. In embodiments, the concave shape of the angled walls of the nozzle structure 172 can be configured to force the wash fluid 174 from the wash channel outlet 170 into the bottom of the flow shaping cavity 173, 273 and to exit though the nozzle outlet 175 (e.g., the reducing portion) with a greater flow velocity than which the wash fluid entered the wash channel 166. The flow velocity of the wash flow exiting the nozzle outlet 175 can be such that the nozzle structure shapes the wash fluid into a fanned spray that can impinge on the imaging device 116 or wipe across the imaging device 116 with enough force to wash bodily fluid (e.g., fat, blood, etc.) from the imaging device 116. In embodiments, the wash channel can be configured to be filled from a syringe at the wash channel inlet 168 to provide wash fluid as needed and on demand during the laparoscopic procedure.

While the insufflation channel 158 is shown radially outward on the radial extension 112 of the nozzle outlet 175, the insufflation channel 158 may not being actively supplying insufflation gas to the surgical cavity 148 at the same time wash fluid is provided to the wash channel 166. Further, the insufflation channel can be located at any suitable location within the longitudinal portion 110, e.g., relative to the wash channel, and the insufflation channel outlet 164 may be located at any suitable location on the radial extension 112. Similarly, the placement of the wash channel outlet 170 and nozzle structure 172 can be any suitable location on the radial extension but can be dependent upon the location of the imaging device 116 within the radial extension 112. In embodiments, the imaging device lens 176 can have a relatively low coefficient of friction such that the wash fluid can easily remove particulate that has accumulated on the imaging device lens.

In accordance with at least one aspect of this disclosure, a kit can include an access port (e.g., access port 104) as described herein and a surgical instrument (e.g., instrument 102) as described herein for inserted into the access port. The access port and the surgical instrument can be included in a sterile package.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the apparatus and methods of the subject disclosure have been shown and described, those skilled in the art will readily appreciate that changes and/or modifications may be made thereto without departing from the scope of the subject disclosure.

What is claimed is:

1. A surgical access device assembly for performing laparoscopic surgery, comprising:
a first surgical instrument;
an access port defining a body, the body having a central bore defined therethrough configured to receive the first surgical instrument,
wherein the first surgical instrument includes:
a longitudinal portion extending along a longitudinal axis of the first surgical instrument and a radial extension extending from a distal end of the longitudinal portion;
an imaging device disposed in the radial extension configured to image a surgical site;
a wash channel disposed in and/or defined through the longitudinal portion and in fluid communication between a wash channel inlet and a wash channel outlet to allow a wash flow to flow from the wash channel inlet to the wash channel outlet; and
a wash nozzle structure radially inward of the imaging device and defining a flow shaping cavity and a wash nozzle outlet in fluid communication with the wash channel outlet, the wash nozzle structure configured to direct the wash flow wash fluid from the wash channel outlet, through the flow shaping cavity, to the nozzle outlet to shape the wash flow to effuse from the wash nozzle outlet such that the wash flow is shaped to sweep across the imaging device to wash the imaging device during the laparoscopic procedure.

2. The assembly of claim 1, wherein the wash channel outlet exits into the flow shaping cavity of the wash nozzle structure.

3. The assembly of claim 2, wherein the wash nozzle structure is configured to increase a flow velocity of the wash flow at the wash nozzle outlet and shape the wash fluid to effuse from the wash nozzle structure at least partially along a spray axis, wherein the spray axis is angled relative to an axis of the radial extension.

4. The assembly of claim 3, wherein the wash nozzle structure includes a nozzle structure body defining a central axis, a proximal face, and a distal face, and a bowl recessed into proximal face of the body, wherein the bowl includes a first angled face defined in a first plane, a second angled face defined in a second plane, a third angled face defined in a third plane, and a fourth face defined in a fourth plane co-planar with the proximal face and the distal face, wherein the first plane, second plane, third plane, and fourth plane are different from one another, wherein the first angled face, the second angled face, and the third angled face are arranged to funnel the wash fluid to the fourth face to direct the wash fluid to spray at least partially along a spray axis.

5. The assembly of claim 4, wherein each of the first angled face, the second angled face and the third angled face have a concave curvature.

6. The assembly of claim 1, wherein the first surgical instrument further includes, an insufflation channel disposed in and/or defined through the longitudinal portion and in fluid communication between an insufflation channel inlet and an insufflation channel outlet to allow insufflation gas to flow from the insufflation channel inlet to the insufflation channel outlet and into a patients abdominal cavity.

7. The assembly of claim 1, wherein the first surgical instrument further includes a lighting device disposed at a distal end of the radial extension, radially outward of the imaging device, configured to illuminate the surgical site within a surgical cavity.

8. The assembly of claim 7, wherein the lighting device further includes at least one illuminator.

9. The assembly of claim 8, wherein the at least one illuminator includes an LED.

10. The assembly of claim 1, wherein the access port further includes an access slot defined in the body configured to receive a second surgical instrument, wherein the second surgical instrument is one of: a laser device, a measuring device, a laser measuring device, a signal transmitting device, a signal receiving device, a signal processing device, a memory storage device, a wiring device, a servo driven device, a gear device, an irrigation device, and/or a suction device.

11. A wash nozzle structure for diverting a wash fluid to a surgical instrument during a laparoscopic procedure, the wash nozzle structure configured to direct the wash fluid to spray along at least partially along a spray axis, wherein the spray axis is angled relative to a plane of a lens of an imaging device of the surgical instrument.

12. The wash nozzle structure of claim 11, wherein the wash nozzle structure includes a nozzle structure body defining a central axis, a proximal face, and a distal face, and a bowl recessed into proximal face of the body, wherein the bowl includes a first angled face defined in a first plane, a second angled face defined in a second plane, a third angled face defined in a third plane, and a fourth face defined in a fourth plane co-planar with the proximal face and the distal face, wherein the first plane, second plane, third plane, and fourth plane are different from one another, wherein the first angled face, the second angled face, and the third angled face are arranged to funnel the wash fluid to the fourth face to direct the wash fluid to spray at least partially along a spray axis.

13. The wash nozzle structure of claim 12, wherein each of the first angled face, the second angled face and the third angled face have a concave curvature.

14. The wash nozzle structure of claim 12, wherein the first angled face and the third angled face are angled relative to the fourth face at a 90 degree angle.

15. The wash nozzle structure of claim 12, wherein the first angled face and the third angled face are angled relative to the fourth face at an angle greater than 90 degrees.

16. The wash nozzle structure of claim 12, wherein the second angled face is angled relative to the fourth face at an angle greater than 90 degrees.

17. The wash nozzle structure of claim 12, wherein the first angled face and the third angled face are angled relative to the second angled face at a 90 degree angle.

18. The wash nozzle structure of claim 12, wherein the first angled face and the third angled face are angled relative to the second angled face at an angle greater than 90 degrees.
